SEQUENCE LISTING (21 sequences)

SEQ ID NO: 1 - protein, 105 aa, Artificial Sequence
  Description of Artificial Sequence/note = Synthetic Construct
MKGLSPIAKGRKTSVSAAVLVSTTIPISSVWGPLQILGQKRGQKMEQANHPVGLDISVVYKDTLKKIVQQETSCPFTHVHYAEGITGRHTAPEDEGSLAQKPPIR

SEQ ID NO: 2 - protein, 270 aa, Artificial Sequence [sequence omitted]

SEQ ID NO: 3 - DNA, 315 bp, Artificial Sequence
  Description of Artificial Sequence/note = Synthetic Construct
atgaagggattatcgcctatcgccaaggggaggaaaactagtgtttctgctgctgtgttggtgagcacaactattccgatcagcagtgtctggggaccattgcagattcttgggcaaaagagaggacagaaaatggagcaggccaatcacccagtggggcttgatatcagtgtggtttacaaggacaccttaaaaaagattgtccaacaagaaacaagctgccccttcacccatgtccactatgctgagggaatcactggaaggcacactgccccagaggatgaaggttctctggcccagaagcccccaatcaga

SEQ ID NO: 4 - DNA, 810 bp, Artificial Sequence
  Description of Artificial Sequence/note = Synthetic Construct
atgaacatcgatgcaaaaatcctcaataaaatactggcaaaccaaatccagcagcacatcaaaaagcttatccaccatgatcaagtgggcttcatccctgggatgcaaggctggttcaacatacacaaatcaataaatgtaatccagcatataaacagaaccaaagacaaaaaccacatgattatctcagtagatgcagaaaaggcctttgacaaagttcaacaacacttcatgctaaaaactctcaataaattaggtattgatgggacgtatctcaaaataataagagctatctatgacaaacccacagccaatatcatactgaatgggctaaaactggaagcattccctttgaaaactggcacaagacagggatgccctctctcacttctcctattcaacatagtgttggaagttctggccagggcaatcaggcaggagaaggaaataaattgtattcaattaggaaaagaggaagttaaattgcccctgtttgcagatgacatgattgtatatctggaaaaccccgtcgtctcagccccaaatctccttaagctgataagcaacttcagcaaagtctcaggatacaaaatcaacgtgcaaaaatcacaagcattcttatacaccaataacagacaaacagagagccaaatcatgagtgaactcccattcacaattgcttcaaagagaataaaatacctaggaatccaacttacaagggatgtgaaggacctcttcaaggagaactacaaaccactgctcaacgaaataaaagaggatacaaacaaatgcaagaacattccatgctca

SEQ ID NO: 5 - DNA, 1263 bp, Artificial Sequence
  Description of Artificial Sequence/note = Synthetic Construct
catctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctaccagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctggaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgagaccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaataga

SEQ ID NO: 6 - DNA, 10 bp, Artificial Sequence
  Description of Artificial Sequence/note = Synthetic Construct
cagagcctgt

SEQ ID NO: 7 - DNA, 10 bp, Artificial Sequence
  Description of Artificial Sequence/note = Synthetic Construct
ctctgggaca

SEQ ID NO: 8 - protein, 375 aa, Artificial Sequence
  Description of Artificial Sequence/note = Synthetic Construct
MKGLSPIAKGRKTSVSAAVLVSTTIPISSVWGPLQILGQKRGQKMEQANHPVGLDISVVYKDTLKKIVQQETSCPFTHVHYAEGITGRHTAPEDEGSLAQKPPIRMNIDAKILNKILANQIQQHIKKLIHHDQVGFIPGMQGWFNIHKSINVIQHINRTKDKNHMIISVDAEKAFDKVQQHFMLKTLNKLGIDGTYLKIIRAIYDKPTANIILNGLKLEAFPLKTGTRQGCPLSLLLFNIVLEVLARAIRQEKEINCIQLGKEEVKLPLFADDMIVYLENPVVSAPNLLKLISNFSKVSGYKINVQKSQAFLYTNNRQTESQIMSELPFTIASKRIKYLGIQLTRDVKDLFKENYKPLLNEIKEDTNKCKNIPCS

SEQ ID NO: 9 - DNA, 1125 bp, Artificial Sequence
  Description of Artificial Sequence/note = Synthetic Construct
atgaagggattatcgcctatcgccaaggggaggaaaactagtgtttctgctgctgtgttggtgagcacaactattccgatcagcagtgtctggggaccattgcagattcttgggcaaaagagaggacagaaaatggagcaggccaatcacccagtggggcttgatatcagtgtggtttacaaggacaccttaaaaaagattgtccaacaagaaacaagctgccccttcacccatgtccactatgctgagggaatcactggaaggcacactgccccagaggatgaaggttctctggcccagaagcccccaatcagaatgaacatcgatgcaaaaatcctcaataaaatactggcaaaccaaatccagcagcacatcaaaaagcttatccaccatgatcaagtgggcttcatccctgggatgcaaggctggttcaacatacacaaatcaataaatgtaatccagcatataaacagaaccaaagacaaaaaccacatgattatctcagtagatgcagaaaaggcctttgacaaagttcaacaacacttcatgctaaaaactctcaataaattaggtattgatgggacgtatctcaaaataataagagctatctatgacaaacccacagccaatatcatactgaatgggctaaaactggaagcattccctttgaaaactggcacaagacagggatgccctctctcacttctcctattcaacatagtgttggaagttctggccagggcaatcaggcaggagaaggaaataaattgtattcaattaggaaaagaggaagttaaattgcccctgtttgcagatgacatgattgtatatctggaaaaccccgtcgtctcagccccaaatctccttaagctgataagcaacttcagcaaagtctcaggatacaaaatcaacgtgcaaaaatcacaagcattcttatacaccaataacagacaaacagagagccaaatcatgagtgaactcccattcacaattgcttcaaagagaataaaatacctaggaatccaacttacaagggatgtgaaggacctcttcaaggagaactacaaaccactgctcaacgaaataaaagaggatacaaacaaatgcaagaacattccatgctca

SEQ ID NO: 10 - DNA, 20 bp, Artificial Sequence
  Description of Artificial Sequence/note = Synthetic Construct
tgagcacaactattccgatc

SEQ ID NO: 11 - DNA, 23 bp, Artificial Sequence
  Description of Artificial Sequence/note = Synthetic Construct
aagcaacttcagcaaagtctcag

SEQ ID NO: 12 - DNA, 21 bp, Artificial Sequence
  Description of Artificial Sequence/note = Synthetic Construct
aaaccactgctcaacgaaata

SEQ ID NO: 13 - DNA, 21 bp, Artificial Sequence
  Description of Artificial Sequence/note = Synthetic Construct
aagggattatcgcctatcgcc

SEQ ID NO: 14 - DNA, 10 bp, Artificial Sequence
  Description of Artificial Sequence/note = Synthetic Construct
ccgcatctac

SEQ ID NO: 15 - DNA, 20 bp, Artificial Sequence
  Description of Artificial Sequence; note = synthetic construct
actcgtgttgataaggctag

SEQ ID NO: 16 - DNA, 23 bp, Artificial Sequence
  Description of Artificial Sequence/note = synthetic construct
ttcgttgaagtcgtttcagagtc

SEQ ID NO: 17 - DNA, 20 bp, Artificial Sequence
  Description of Artificial Sequence/note = synthetic construct
ctcgtgttgataaggctagt

SEQ ID NO: 18 - DNA, 20 bp, Artificial Sequence
  Description of Artificial Sequence/note = synthetic construct
tcgtgttgataaggctagtc

SEQ ID NO: 19 - DNA, 20 bp, Artificial Sequence
  Description of Artificial Sequence/note = synthetic construct
cgtgttgataaggctagtcg

SEQ ID NO: 20 - DNA, 23 bp, Artificial Sequence
  Description of Artificial Sequence/note = synthetic construct
tcgttgaagtcgtttcagagtcc

SEQ ID NO: 21 - DNA, 22 bp, Artificial Sequence
  Description of Artificial Sequence/note = synthetic construct
gttgaagtcgtttcagagtcct

(19) United States
(12) Patent Application Publication
(10) Pub. No.: US 2007/0254283 A1
(43) Pub. Date: Nov. 1, 2007

(54) Brhf1 as a Cancer Diagnostic Marker

(76) Inventor: Roy Deudutta, Miramar, FL (US)

Correspondence Address: NEEDLE & ROSENBERG, P.C., SUITE 1000, 999 PEACHTREE STREET, ATLANTA, GA, 30309-3915, US

(21) Appl. No.: 10/591,726

(22) PCT Filed: Mar. 4, 2005
(86) PCT No.: PCT/US05/06950

§ 371 Date: Jun. 5, 2007

Related U.S. Application Data
(60) Provisional application No. 60/550,224, filed on Mar. 4, 2004.

(51) Int. Cl. C07H 21/04; C07K 14/00; C12N 1/20; C12N 15/00; C12N 5/00; C12Q 1/68; G01N 33/53

(52) U.S. Cl. 435/6; 435/252.8; 435/320.1; 435/325; 435/375; 435/7.8; 530/350; 530/387.1; 536/23.5

(57) ABSTRACT

This invention relates generally to the discovery of a novel gene, designated BRHF1. The invention further relates to the detection of the BRHF1 gene, BRHF1 expression and/or the presence of the BRHF1 protein as potential markers for the early diagnosis of cancer.

This application claims priority to U.S. provisional application Ser. No. 60/550,224, filed Mar. 4, 2005, which is herein incorporated by this reference in its entirety.

### FIELD OF THE INVENTION

This invention relates generally to the discovery of a novel gene, designated BRHF1. The invention further relates to the detection of the BRHF1 gene, BRHF1 expression and/or the presence of the BRHF1 protein as markers for the early diagnosis of cancer.

### SUMMARY OF THE INVENTION

The present invention provides purified BRHF1 polypeptides and nucleic acids encoding these polypeptides.

Also provided by the present invention are antibodies that specifically bind to BRHF1 polypeptides.

Further provided by the present invention is a method for detecting the presence of cancer in a subject, comprising: (a) contacting a biological sample obtained from a subject with at least two oligonucleotide primers, each primer consisting of 10 to 200 contiguous nucleotides of SEQ ID NO: 5 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of cancer in the subject.

Also provided by the present invention is a method for detecting the presence of cancer in a subject, comprising: (a) contacting a biological sample obtained from a subject with a nucleic acid probe under conditions that allow the probe to selectively bind a BRHF1 nucleic acid; and (b) detecting the presence of a BRHF1 nucleic acid, whereby the presence of a BRHF1 nucleic acid indicates the presence of cancer in the subject.

Further provided by the present invention is a method of detecting the presence of cancer in a subject comprising: a) contacting a sample from the subject with an antibody to a BRHF1 polypeptide; and b) detecting the antibody bound to the BRHF1 polypeptide in the sample, wherein binding of BRHF1 polypeptide to the antibody indicates the presence of a BRHF1 polypeptide in the sample, the presence of a BRHF1 polypeptide indicating the presence of cancer in the subject.

Also provided is a method of reducing BRHF1 expression in a cell comprising administering to the cell an antisense oligonucleotide that specifically binds to mRNA transcribed from the BRHF1 gene under conditions that allow hybridization, wherein the BRHF1 mRNA comprises a nucleotide sequence selected from the group consisting of SEQ ID NO: 3, SEQ ID NO: 4 and SEQ ID NO: 9 and wherein hybridization of the antisense oligonucleotide with the BRHF1 mRNA reduces BRHF1 expression.

The present invention also provides a method of reducing BRHF1 expression comprising administering to a cell a ribozyme that specifically binds to mRNA transcribed from the BRHF1 gene, the ribozyme binding reducing BRHF1 expression.

Also provided by the present invention is a method of reducing BRHF1 expression comprising administering to a cell an siRNA that is complementary to at least a portion of the coding sequence of BRHF1, under conditions that allow hybridization of the siRNA with the BRHF1 coding sequence, wherein the BRHF1 coding sequence comprises a nucleotide sequence selected from the group consisting of SEQ ID NO: 3 SEQ ID NO: 4 and SEQ ID NO: 9 and wherein the binding of the siRNA to the BRHF1 coding sequence reduces BRHF1 expression.

Further provided by the present invention is a method of identifying a compound that reduces BRHF1 expression, comprising administering a test compound to a cell containing a BRHF1 gene and detecting the level of the BRHF1 gene product produced, a decrease in the gene product as compared to a control cell indicating the compound reduces BRHF1 expression.

Also provided is a method of identifying a compound that reduces BRHF1 expression, comprising administering a test compound to a cell containing a nucleic acid encoding the polypeptide of claim 1 and detecting the level of the BRHF1 gene product produced, a decrease in the gene product indicating a compound that reduces BRHF1 expression.

The present invention also provides a method of identifying a compound that reduces BRHF1 expression in the presence of estrogen, comprising administering a test compound and estrogen to a cell containing a BRHF1 gene and detecting the level of the BRHF1 gene product produced, a decrease in the gene product as compared to a control cell indicating a compound that reduces BRHF1 expression in the presence of estrogen.

Also provided is a method of identifying a compound that reduces BRHF1 expression in the presence of estrogen, comprising administering a test compound and estrogen to a cell containing a nucleic acid encoding the polypeptide of claim 1 and detecting the level of the BRHF1 gene product produced, a decrease in the gene product indicating a compound that reduces BRHF1 expression in the presence of estrogen.

### DESCRIPTION OF THE FIGURES

FIG. 1 shows the AP-PCR fingerprints of matched uninvolved breast tissue (N) and tumor (T) tissue DNA using OPC04 (5′CCGCATCTAC3′) (SEQ ID NO: 14) primer. The amplification product was resolved on denaturing urea-sequencing gel and detected by silver staining. The two arrows on the right side of the photograph indicates the two single strand of 1270 bp amplification product amplified in tumors.

FIG. 2 illustrates chromosomes in metaphase showing the chromosomal localization of BRHF1 gene on 17q11.2 (Left panel). Two ideograms illustrating the chromosomal position of BRHF1 gene at 17q11.2 are also provided. (Right panel).

FIG. 3A is a Northern blot analysis of the BRHF-1 gene demonstrating a transcript size of 6.5 kb (indicated by arrow).

FIG. 3B shows the that the expected 6.5 kb BRHF1 gene transcript was detected by Northern blot only from RNA of MCF7(ER+ve) cells, and not from MDA-MB 468(ER−ve) cells.

FIG. 4 is an agarose gel photograph of the BRHF1 gene transcript detected by RT-PCR analysis. Multifold higher level of BRHF1 gene transcript (indicated by arrow) was observed in tumors (T) as compared to matched normal samples. Equal intensity of β-actin gene amplified confirmed the same amount of RNA taken from N and T for cDNA synthesis.

FIG. 5 is a RT-PCR analysis of the BRHF1 gene transcript with the RNA isolated from three batches (ET1, ET₂, ET₃) of MCF-7 cells treated with 100 pg/ml of 17 β-estradiol, and their corresponding untreated controls (C₁, C₂, C₃). Higher levels of BRHF1 gene expression (indicated by an arrow) were observed in estrogen-treated MCF7 cells.

FIG. 6 is an ORF region encoding 1275 amino acids representing a part of a novel gene of the BRHF-1 gene-family located on human chromosome Xp22. The insert DNA sequence (SEQ ID NO: 5) of BRHF1 found on chromosome 17 of the present invention is underlined; it contains FG repeats and nuclear localization signals KKKR; and shows significant similarity with neucleoporin FG repeat containing family proteins. Amino acid sequences in italics represents a region showing significant similarity with a conserved domain of an Exo_endo_phos family protein, and amino acid sequences in bold represent a conserved domain of the reverse transcriptase family protein.

### DETAILED DESCRIPTION OF THE INVENTION

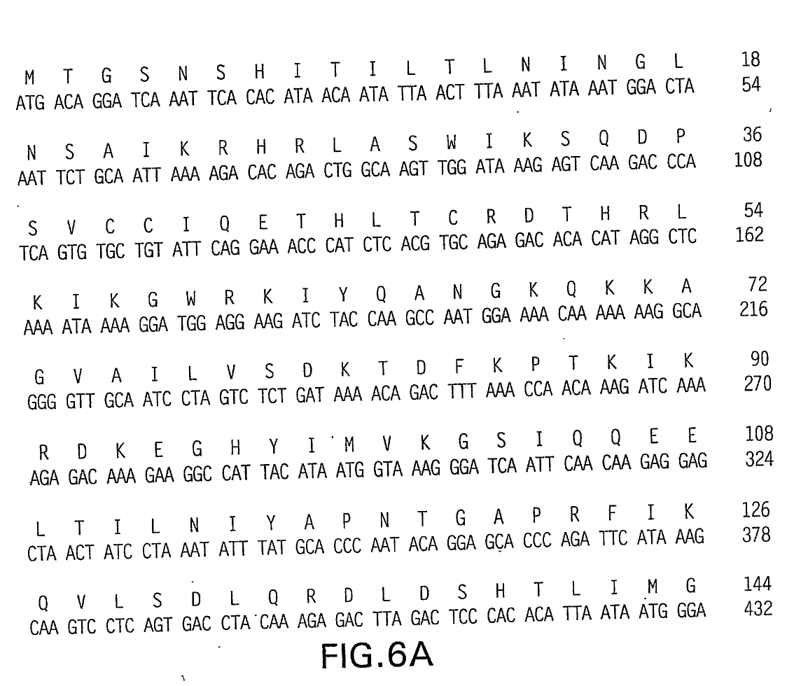

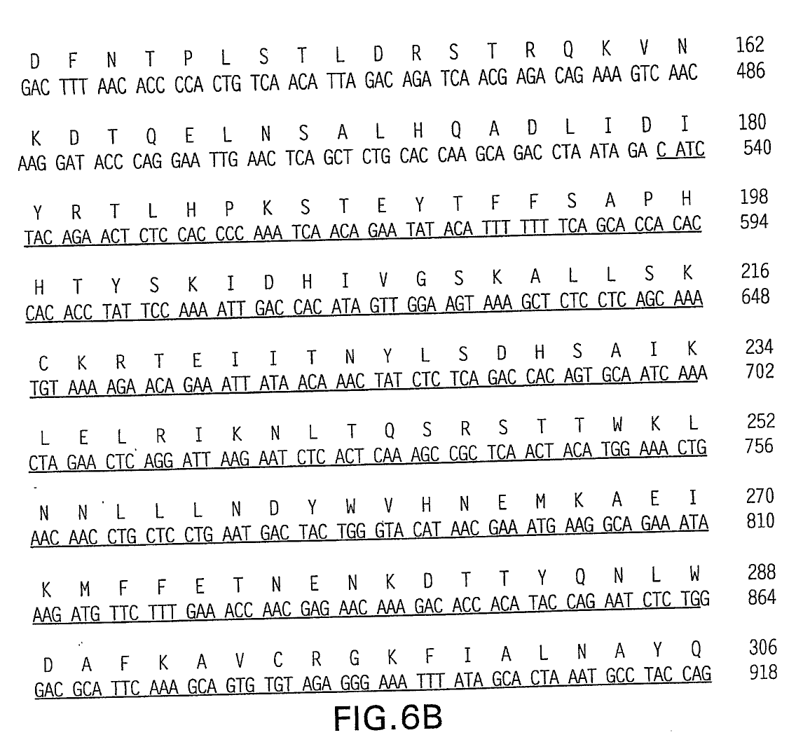

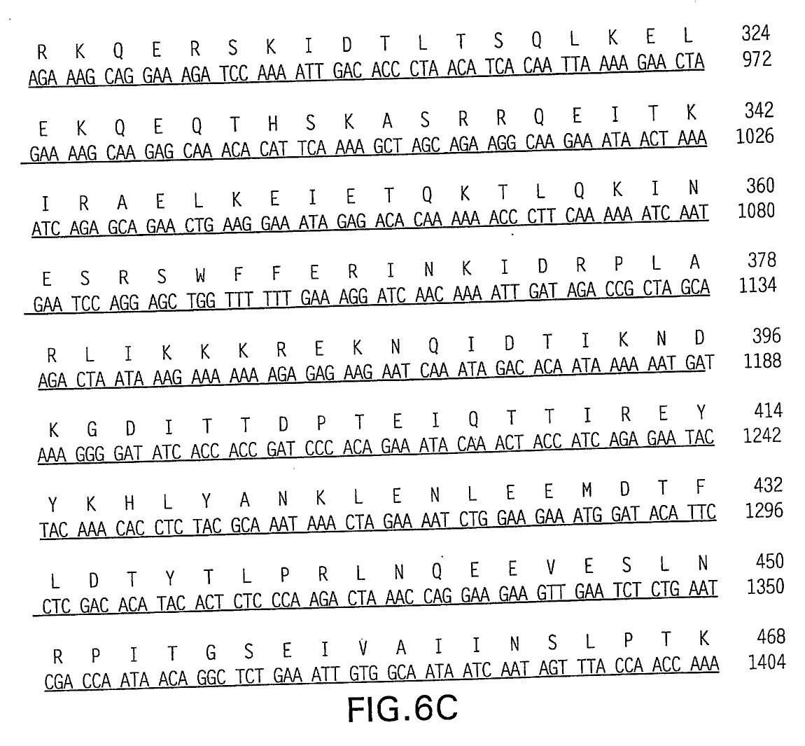

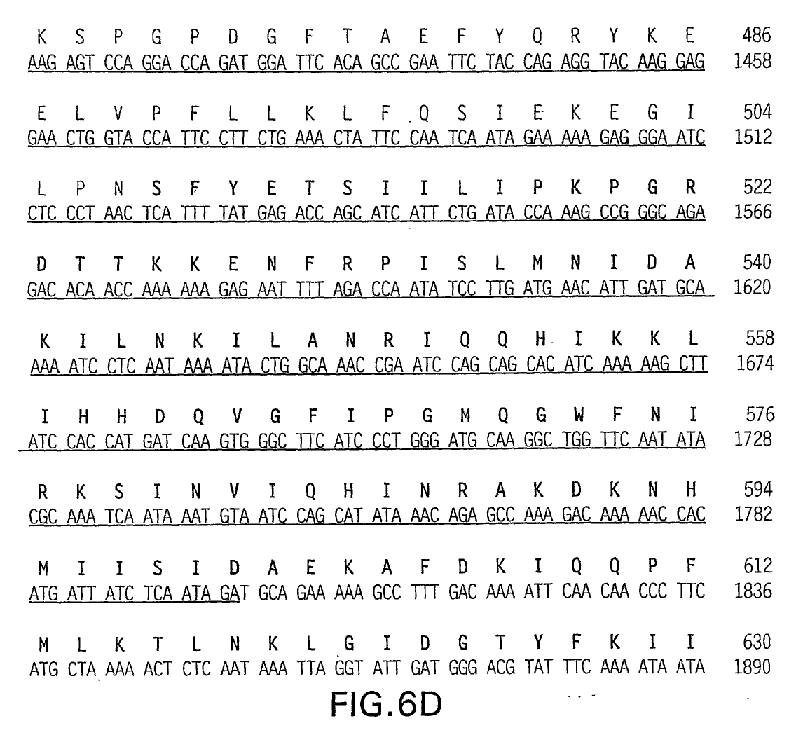

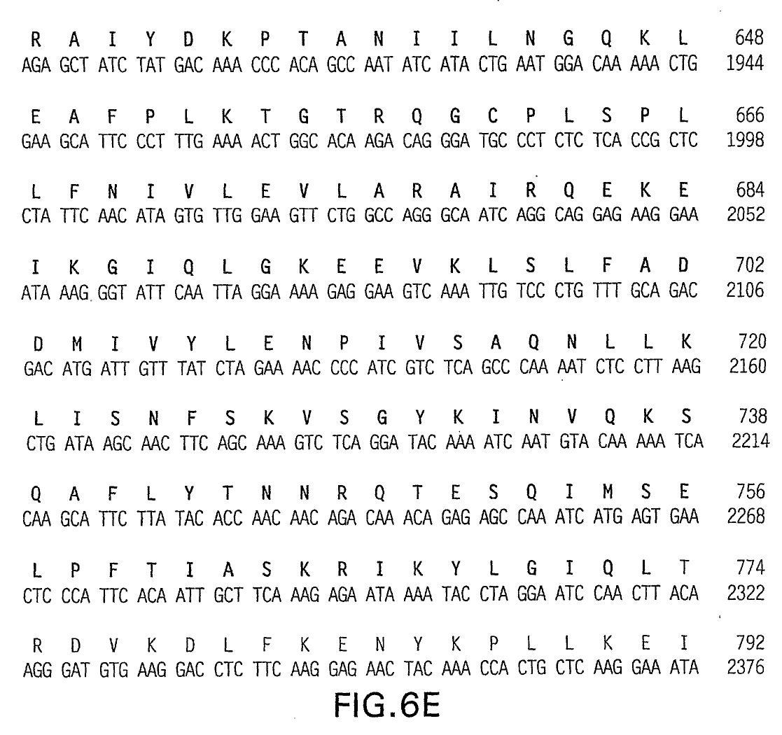

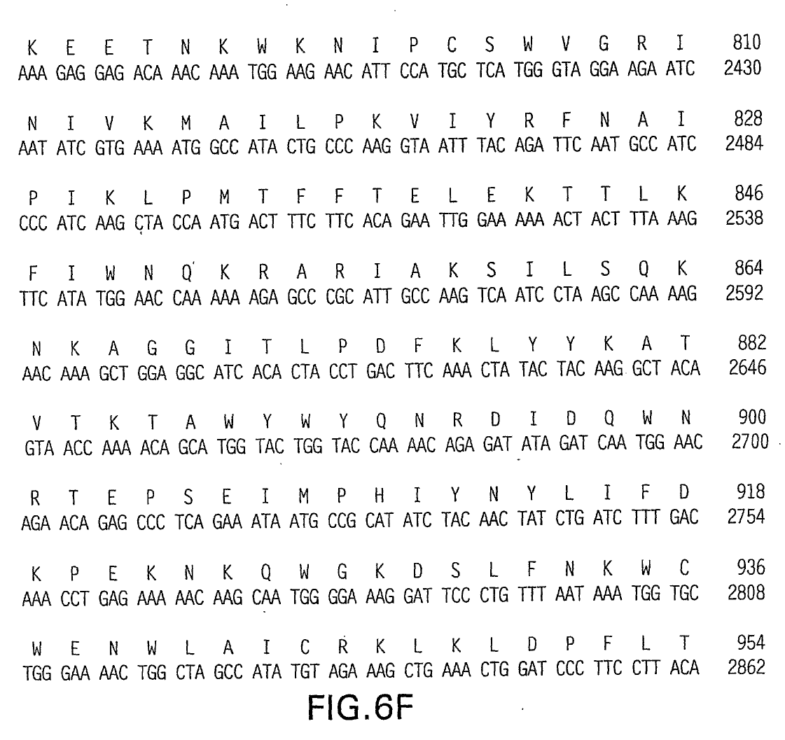

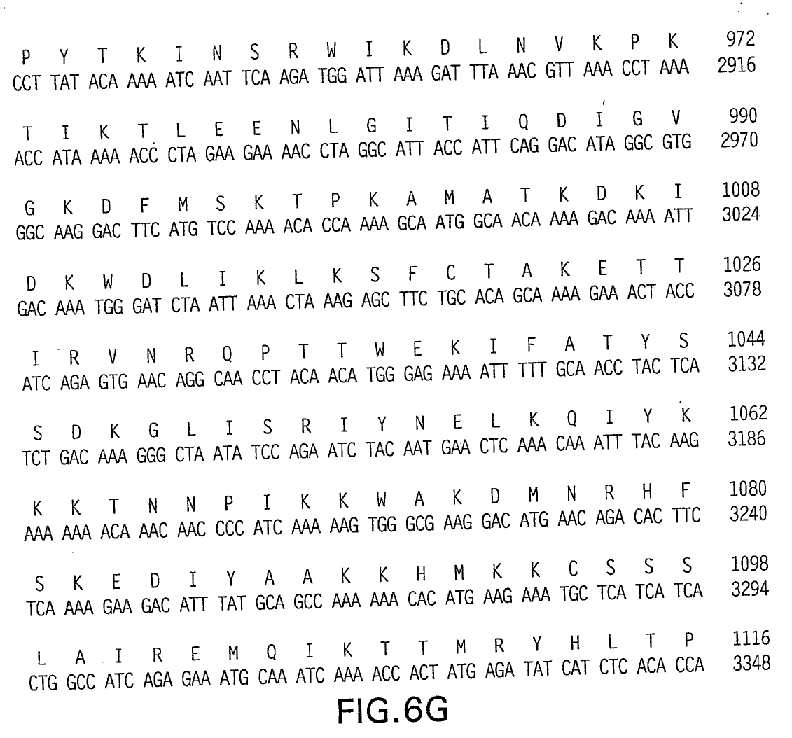

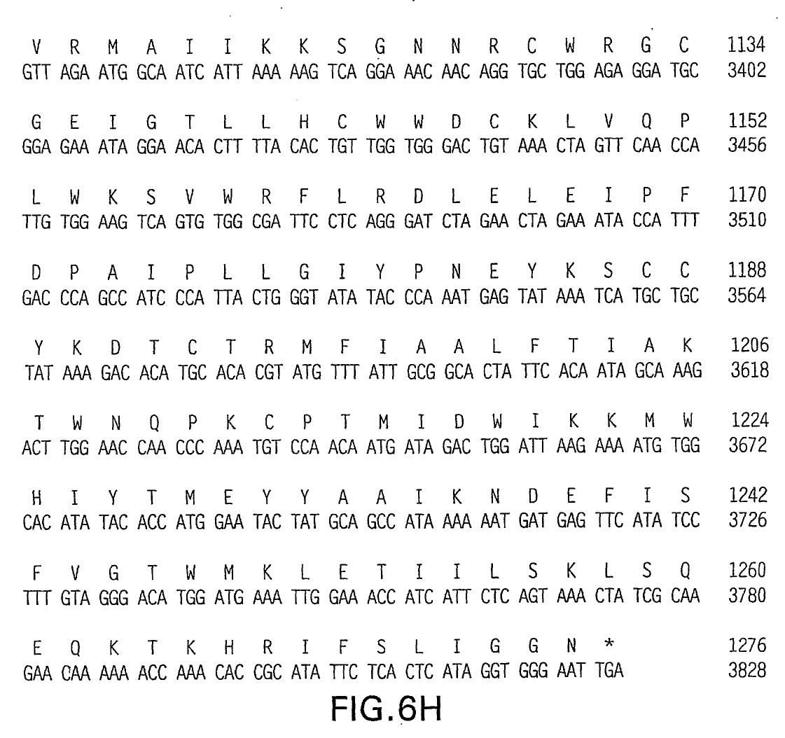

Using Arbitrary-Primed PCR (AP-PCR), mutations were found in over 80% of breast tumor samples analyzed as compared with the uninvolved breast tissue from the same subject. Further characterization by cloning and sequencing of this genomic region harboring mutation revealed that it represents a part of a novel gene named BRHF1. The mutation present in breast tumor samples is a 1270 bp insertion located in chromosomal region 17q11.2. Thus, applicants have provided the genomic locus corresponding to this insertion. The insert located in chromosomal region 17q11.2 comprises SEQ ID NO: 5 which is also the underlined sequence set forth in FIG. 6. Therefore, the present invention provides methods of detecting the genomic region harboring this mutation as well as methods of detecting BRHF1 transcripts and fragments thereof. Also provided are methods of detecting a BRHF1 polypeptide. These methods can be utilized to detect, stage and treat cancer in a subject.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “an antibody” includes mixtures of antibodies, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. The term “subject” includes domesticated animals, such as cats, dogs, etc., livestock (e.g., cattle, horses, pigs, sheep, goats, etc.), and laboratory animals (e.g., mouse, rabbit, rat, gerbil, guinea pig, etc.).

BRHF1 Polypeptides

The present invention provides an isolated polypeptide comprising SEQ ID NO: 1. SEQ ID NO: 1 corresponds to amino acid residues 1-105 of a full-length BRHF1 polypeptide. The present invention also provides an isolated polypeptide comprising SEQ ID NO: 2 (amino acid residues 106-375 of a BRHF1 polypeptide). The present invention also provides an isolated polypeptide comprising SEQ ID NO: 8 (amino acid residues 1-375 of a BRHF1 polypeptide) as set forth below:

MKGLSPIAKGRKTSVSAAVLVSTTIPISSVWGPLQILGQKRGQKMEQANG
PVGLDISVVYKDTLKKIVQQETSCPFTHVHYAEGITGRHTAPEDEGSLAQ
KPPIRMNIDAKILNKILANQIQQHIKKLIHHDQVGFIPGMQGWFNIHKSI
NVIQHINRTKDKNHMIISVDAEKAFDKVQQHFMLKTLNKLGIDGTYLKII
RAIYDKPTANIILNGLKLEAFPLKTGTRQGCPLSLLLFNIVLEVLARAIR
QEKEINCIQLGKEEVKLPLFADDMIVYLENPVVSAPNLLKLISNFSKVSG
YKINVQKSQAFLYTNNRQTESQIMSELPFTIASKRIKYLGIQLTRDVKDL
FKENYKPLLNEIKEDTNKCKNIPCS

The present invention also provides fragments of SEQ ID NO: 1, SEQ ID NO: 2 and SEQ ID NO: 8. Optionally, the fragments comprise at least 10, 20, 30, 40, 50, 60, 70, 80, 90, 100, 110, 120, 130, 140 or any number or amino acids less than the full length BRHF1 polypeptide. For example, the fragments of the present invention include but are not limited to fragments comprising amino acids 1-374 of SEQ ID NO: 8, fragments comprising amino acids 1-373 of SEQ ID NO: 8, fragments comprising amino acids 1-372 of SEQ ID NO: 8, fragments comprising amino acids 1-371 of SEQ ID NO: 8, fragments comprising amino acids 1-370 of SEQ ID NO: 8, fragments comprising amino acids 1-369 of SEQ ID NO: 8, fragments comprising amino acids 1-368 of SEQ ID NO: 8, fragments comprising amino acids 1-367 of SEQ ID NO: 8, fragments comprising amino acids 1-366 of SEQ ID NO: 8, fragments comprising amino acids 1-365 of SEQ ID NO: 8, fragments comprising amino acids 1-364 of SEQ ID NO: 8, fragments comprising amino acids 1-300 of SEQ ID NO: 8, fragments comprising amino acids 1-250 of SEQ ID NO: 8, fragments comprising amino acids 1-200 of SEQ ID NO: 8, fragments comprising amino acids 1-150 of SEQ ID NO: 8, fragments comprising amino acids 1-100 of SEQ ID NO: 8, fragments comprising amino acids 1-50 of SEQ ID NO: 8 etc. Such fragments include one or more conservative amino acid substitutions as compared to the sequence of SEQ ID NO: 8. Optionally, the fragments have at least 75%, 80%, 85%, 90%, 95% 99% identity or any percentage in between as compared to the corresponding position of SEQ ID NO: 8.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, polypeptide may be obtained by cleaving full length polypeptides. When the polypeptide is a fragment of a larger naturally occurring polypeptide, the isolated polypeptide is shorter than and excludes the full-length, naturally-occurring polypeptide of which it is a fragment.

The polypeptides of the invention can be prepared using any of a number of chemical polypeptide synthesis techniques well known to those of ordinary skill in the art including solution methods and solid phase methods. One method of producing the polypeptides of the present invention is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin, whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The polypeptides of the invention can also be prepared by other means including, for example, recombinant techniques. Examples of appropriate cloning and sequencing techniques, and instructions sufficient to direct persons of skill through many cloning exercises are found in Sambrook et al. (2001) Molecular Cloning—A Laboratory Manual (3rd ed.) Vol. 1-3, Cold Spring Harbor Laboratory, Cold Spring Harbor Press, NY, (Sambrook).

Also provided by the present invention is a polypeptide comprising an amino acid sequence at least about 70%, 75%, 80%, 85%, 90%, 95%, 96%, 97%, 98% or 99% identical to a sequence selected from the group consisting of: SEQ ID NO: 1, SEQ ID NO: 2 and SEQ ID NO: 8.

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. These variants can be naturally occurring variants of the BRHF1 sequences disclosed herein, for example polymorphic variants of BRHF1. The present invention also provides homologs of BRHF1, for example, a mouse homolog, a rat homolog, a pig homolog, a monkey homolog etc. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Also provided by the present invention is a polypeptide comprising an amino acid sequence selected from the group consisting of: SEQ ID NO: 1, SEQ ID NO: 2 and SEQ ID NO: 8, or fragments thereof, with one or more conservative amino acid substitutions. These conservative substitutions are such that a naturally occurring amino acid is replaced by one having similar properties. Such conservative substitutions do not alter the function of the polypeptide. For example, conservative substitutions can be made according to the following table:

TABLE 1
Amino Acid Substitutions

| Original Residue | Exemplary Substitutions |
|---|---|
| Arg | Lys |
| Asn | Gln |
| Asp | Glu |
| Cys | Ser |
| Gln | Asn |
| Glu | Asp |
| Gly | Pro |
| His | Gln |
| Ile | leu; val |
| Leu | ile; val |
| Lys | arg; gln |
| Met | leu; ile |
| Phe | met; leu; tyr |
| Ser | Thr |
| Thr | Ser |
| Trp | Tyr |
| Tyr | trp; phe |
| Val | ile; leu |

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity.

Further provided by the invention are antigenic fragments of SEQ ID NO: 1, SEQ ID NO: 2 and SEQ ID NO: 8. It is also understood that a BRHF1 gene product, such as the BRHF1 polypeptides comprising SEQ ID NO: 1, SEQ ID NO: 2 or SEQ ID NO: 8 or fragments thereof, can exert a biological function associated with the BRHF1 gene. An active BRHF1 gene product may act as a transcriptional activator to elicit downstream effects. The BRHF1 gene product may also function from outside of the cell either as a ligand which can bind to its cell surface receptor and participate in signal transduction, or it may function through the interaction with other secreted proteins and extracellular matrix molecules outside of cells. A BRHF1 gene product could also be involved in cell proliferation, metastasis and other cancer-associated processes. Any effect associated with BRHF1 associated cancers is also contemplated by this invention.

Nucleic Acids

The present invention also provides nucleic acids that encode BRHF1 polypeptides. The present invention also provides a nucleic acid encoding a polypeptide comprising a sequence selected from the group consisting of: SEQ ID NO: 1, SEQ ID NO: 2 and SEQ ID NO: 8. The present invention also provides nucleic acid encoding a polypeptide comprising a sequence selected from the group consisting of: a fragment of SEQ ID NO: 1, a fragment of SEQ ID NO: 2 and a fragment of SEQ ID NO: 8. The present invention also provides an isolated nucleic acid sequence comprising a nucleic acid sequence corresponding to a genomic insertion (SEQ ID NO: 5) of approximately 1270 bp found in the mutated BRHF1 gene associated with cancer. The present invention further provides isolated genomic sequences comprising SEQ ID. NO: 5. Also provided are nucleic acid sequences that encode fragments of BRHF1 polypeptides as disclosed herein. The nucleic acids of this invention can be isolated. As used herein, the term “isolated” refers to a nucleic acid separated or significantly free from at least some of the other components of the naturally occurring organism, for example, the cell structural components commonly found associated with nucleic acids in a cellular environment and/or other nucleic acids. The nucleic acids of this invention can be isolated from cells according to any of many methods well known in the art.

An example of a nucleic acid encoding a polypeptide comprising SEQ ID NO: 1, is provided herein as SEQ ID NO: 3. An example of a nucleic acid encoding a polypeptide comprising SEQ ID NO: 2, is provided herein as SEQ ID NO: 4. An example of a nucleic acid encoding a polypeptide comprising SEQ ID NO: 8 is provided herein as SEQ ID NO: 9.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system.

The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in Sambrook et al., “Molecular Cloning, a Laboratory Manual,” Cold Spring Harbor Laboratory Press (1989).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

Vectors, Cells, and Methods of Using

Also provided is a vector, comprising a nucleic acid of the present invention. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification. The vector can comprise the nucleic acid in pET15b, pSRα-Neo, pPICZα, or pPIC9K.

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae. There are several advantages to yeast expression systems, which include, for example, Saccharomyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFα-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a myeloma cell, a Pichia cell, or an insect cell. The coding sequence for any of the polypeptides described herein can be introduced into a Chinese hamster ovary (CHO) cell line, for example, using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines have been developed and include myeloma cell lines, fibroblast cell lines, and a variety of tumor cell lines such as melanoma cell lines. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

The present invention provides a method of making any of the polypeptides derived from the BRHF1 polypeptide described herein comprising: culturing a host cell comprising a vector that encodes a BRHF1 polypeptide and purifying the polypeptide produced by the host cell. As mentioned above, these polypeptides include, but are not limited to, a polypeptide comprising SEQ ID NO: 1, SEQ ID NO: 2 or SEQ ID NO: 8. Such polypeptides can also include variants of SEQ ID NO: 1, SEQ ID NO: 2 or SEQ ID NO: 8 or fragments thereof as described herein.

Antibodies

The present invention further provides an isolated antibody or fragment thereof that specifically binds an epitope contained within amino acids 1-105 of the BRHF1 polypeptide. In other words, the present invention provides an isolated antibody or fragment thereof that specifically binds an epitope contained within the amino acid sequence of SEQ ID NO: 1. Further provided by the present invention is an isolated antibody or fragment thereof that specifically binds an epitope contained within amino acids 106-375 of the BRHF1 polypeptide (SEQ ID NO: 2). The present invention also provides an antibody that specifically binds to an epitope contained within or overlapping with amino acids 1-105 of the BRHF1 polypeptide, as evidenced by competitive binding studies. The present invention also provides an antibody that specifically binds to an epitope contained within or overlapping with amino acids 106-375 of the BRHF1 polypeptide, as evidenced by competitive binding studies. Competitive binding studies are well known in the art. For example, if a test antibody competes for binding to the BRHF1 polypeptide with an antibody or ligand that specifically binds an epitope contained within amino acids 1-105 of the BRHF1 polypeptide, one of skill in the art would readily know that the test antibody binds the same epitope as the antibody or ligand that specifically binds to an epitope contained within or overlapping with amino acids 1-105 of the BRHF1 polypeptide. Similarly, if a test antibody competes for binding to the BRHF1 polypeptide with an antibody or ligand that specifically binds an epitope contained within amino acids 106-375 of the BRHF1 polypeptide, one of skill in the art would readily know that the test antibody binds the same epitope as the antibody or ligand that specifically binds to an epitope contained within or overlapping with amino acids 106-375 of the BRHF1 polypeptide.

The antibody of the present invention can be a polyclonal antibody or a monoclonal antibody. The antibody of the invention selectively binds a BRHF1 polypeptide. By “selectively binds” or “specifically binds” is meant an antibody binding reaction which is determinative of the presence of the antigen (in the present case, a BRHF1 polypeptide or antigenic fragments thereof) among a heterogeneous population of proteins and other biologics. Thus, under designated immunoassay conditions, the specified antibodies bind preferentially to a particular peptide and do not bind in a significant amount to other proteins in the sample. Specific binding to a BRHF1 polypeptide under such conditions requires an antibody that is selected for its specificity to a BRHF1 polypeptide. Preferably, selective binding includes binding at about or above 1.5 times assay background and the absence of significant binding is less than 1.5 times assay background.

This invention also contemplates antibodies that compete for binding to natural BRHF1 interactors. For example, an antibody of the present invention can compete with BRHF1 for a binding site (e.g. a receptor) on a cell or the antibody can compete with BRHF1 for binding to another protein or biological molecule. The antibody optionally can have either an antagonistic or agonists function as compared to the antigen. Preferably, the antibody binds a BRHF1 polypeptide ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity matured antibodies are also considered to be part of the invention.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse or other species. The heavy chain constant domains that correspond to the different classes of immunoglobulins are called alpha, delta, epsilon, gamma, and mu, respectively.

As used herein, the terms “immunoglobulin heavy chain or fragments thereof” and “immunoglobulin light chain or fragments thereof” encompass chimeric peptides and hybrid peptides, with dual or multiple antigen or epitope specificities, and fragments, including hybrid fragments. Thus, fragments of the heavy chains and/or fragments of the light chains that retain the ability to bind their specific antigens are provided. For example, fragments of the heavy chains and/or fragments of the light chains that maintain BRHF1 protein binding activity are included within the meaning of the terms “immunoglobulin heavy chain or fragments thereof” and “immunoglobulin light chain and fragments thereof,” respectively. Such heavy chains and light chains and fragments thereof, respectively, can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain BRHF1 protein binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a BRHF1 monoclonal antibody. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is important that antibodies be humanized with retention of high affinity for the antigen and other favorable biological properties. To achieve this goal, according to one method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

The present invention further provides a hybridoma cell that produces the monoclonal antibody of the invention. An example of such a hybridoma cell is a hybridoma cell which produces a monoclonal antibody that specifically binds an epitope contained within amino acids 1-105 of the BRHF1 polypeptide. The present invention further provides a hybridoma cell which produces a monoclonal antibody that specifically binds an epitope contained within amino acids 106-375 of the BRHF1 polypeptide. Also provided by the present invention is a hybridoma cell which produces a monoclonal antibody that specifically binds an epitope contained within amino acids 1-375 of the BRHF1 polypeptide.

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies of the invention may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a polypeptide of the present invention. Thus, the antibodies of the present invention specifically bind BRHF1 protein.

Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of BRHF1 protein, for example, expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by reference in full for the the methods of antibody production).

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing, for example, domains of BRHF1 protein antibody as fusion proteins. The antigen is produced by inserting a gene fragment in-frame between the signal sequence and the mature protein domain, for example, of the BRHF1 protein antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against BRHF1 protein. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody of the invention or substituted for the variable domains of one antigen-combining site of an antibody of the invention to create a chimeric bivalent antibody comprising one antigen-combining site having specificity for BRHF1 polypeptide and another antigen-combining site having specificity for a different antigen.

The present invention further provides an isolated nucleic acid, comprising a nucleotide sequence that encodes an immunoglobulin heavy chain or fragment thereof of an antibody or fragment thereof of an antibody that specifically binds an epitope contained within amino acids 1-105 of the BRHF1 protein (SEQ ID NO: 1), an isolated nucleic acid, comprising a nucleotide sequence that encodes an immunoglobulin heavy chain or fragment thereof of an antibody or fragment thereof of an antibody that specifically binds an epitope contained within amino acids 106-375 of the BRHF1 protein (SEQ ID NO: 2) and an isolated nucleic acid, comprising a nucleotide sequence that encodes an immunoglobulin heavy chain or fragment thereof of an antibody or fragment thereof of an antibody that specifically binds an epitope contained within amino acids 1-375 of the BRHF1 protein (SEQ ID NO: 8).

Vectors comprising any of the nucleic acids comprising a nucleotide sequence that encodes an immunoglobulin heavy chain or a fragment thereof are also contemplated by this invention as are host cells comprising such vectors. Suitable vectors and host cells for expression of the immunoglobulin heavy chains of the present invention are described above.

The present invention further provides an isolated nucleic acid, comprising a nucleotide sequence that encodes an immunoglobulin light chain or fragment thereof of an antibody or fragment thereof of an antibody that specifically binds an epitope contained within amino acids 1-105 of the BRHF1 protein (SEQ ID NO: 1), an isolated nucleic acid, comprising a nucleotide sequence that encodes an immunoglobulin light chain or fragment thereof of an antibody or fragment thereof of an antibody that specifically binds an epitope contained within amino acids 106-375 of the BRHF1 protein (SEQ ID NO: 2) and an isolated nucleic acid, comprising a nucleotide sequence that encodes an immunoglobulin light chain or fragment thereof of an antibody or fragment thereof of an antibody that specifically binds an epitope contained within amino acids 1-375 of the BRHF1 protein (SEQ ID NO: 8).

Vectors comprising any of the nucleic acids comprising a nucleotide sequence that encodes an immunoglobulin light chain or a fragment thereof are also contemplated by this invention as are host cells comprising such vectors. Suitable vectors and host cells for expression of the immunoglobulin light chains of the present invention are described above.

Further provided by this invention is a purified polypeptide, comprising an amino acid sequence of an immunoglobulin heavy chain or a fragment thereof of an antibody that binds an epitope contained within amino acids 1-105 of the BRHF1 protein (SEQ ID NO: 1), a purified polypeptide, comprising an amino acid sequence of an immunoglobulin heavy chain or a fragment thereof of an antibody that binds an epitope contained within amino acids 106-375 of the BRHF1 protein (SEQ ID NO: 2) and a purified polypeptide, comprising an amino acid sequence of an immunoglobulin heavy chain or a fragment thereof of an antibody that binds an epitope contained within amino acids 1-375 of the BRHF1 protein (SEQ ID NO: 8).

Methods of obtaining polypeptides comprising an amino acid sequence of an immunoglobulin heavy chain and polypeptides comprising an amino acid sequence of an immunoglobulin heavy chain are described above.

Further provided by this invention is a purified polypeptide, comprising an amino acid sequence of an immunoglobulin light chain or a fragment thereof of an antibody that binds an epitope contained within amino acids 1-105 of the BRHF1 protein (SEQ ID NO: 1), a purified polypeptide, comprising an amino acid sequence of an immunoglobulin light chain or a fragment thereof of an antibody that binds an epitope contained within amino acids 106-375 of the BRHF1 protein (SEQ ID NO: 2) and a purified polypeptide, comprising an amino acid sequence of an immunoglobulin light chain or a fragment thereof of an antibody that binds an epitope contained within amino acids 1-375 of the BRHF1 protein (SEQ ID NO: 8).

Methods of obtaining polypeptides comprising an amino acid sequence of an immunoglobulin light chain and polypeptides comprising an amino acid sequence of an immunoglobulin light chain are described above.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies of the present invention is to link two or more peptides or polypeptides together by protein chemistry techniques. For example, peptides or polypeptides can be chemically synthesized using currently available laboratory equipment using either Fmoc(9-fluorenylmethyloxycarbonyl) or Boc(tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin, whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The invention also provides fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with BRHF1 protein. For example, amino acids found not to contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of a monoclonal antibody or fragment thereof of the invention and one or more reagents for detecting binding of the antibody or fragment thereof to a BRHF1 polypeptide. The reagents can include, for example, fluorescent tags, enzymatic tags, or other tags. The reagents can also include secondary or tertiary antibodies or reagents for enzymatic reactions, wherein the enzymatic reactions produce a product that can be visualized.

Nucleic Acid Detection Methods

As mentioned above, using Arbitrary-Primed PCR (AP-PCR), mutations were found in over 80% of the breast tumor samples analyzed as compared with the uninvolved breast tissue from the same subject. Further characterization by cloning and sequencing of this genomic region harboring mutation revealed that it represents a part of a novel gene named BRHF1. Applicants subsequently identified the full length sequence of a BRHF1 polypeptide (SEQ ID NO: 3) and the nucleic acid encoding the full length sequence (SEQ ID NO: 9). The mutation present in breast tumor samples is a 1270 bp insert located in chromosomal region 17q11.2. Therefore, the present invention also provides methods of detecting the genomic region harboring this mutation as well as methods of detecting BRHF1 transcripts and fragments thereof. These methods can be utilized to detect cancer in a subject.

The term cancer, when used herein refers to or describes the physiological condition, preferably in a mammalian subject, that is typically characterized by unregulated cell growth. Examples of cancer include but are not limited to ras-induced cancers, colorectal cancer, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, estrogen dependent breast cancer, prostrate carcinoma, rhabdomyosarcoma, colon carcinoma, ovarian cancer and head and neck cancer.

The sample of this invention can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, lung fluid, amnion cells, fresh tissue, dry tissue, and cultured cells or tissue. The sample can be from malignant tissue or non-malignant tissue. The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention. Furthermore, the sample can be embedded in any commercially available mounting medium, either aqueous or organic.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of BRHF1, or BRHF1 nucleic acid of the invention can be on, supported by, or attached to a substrate which facilitates detection. Such a substrate can include a chip, a microarray or a mobile solid support. Thus, provided by the invention are substrates including one or more of the antibodies or antibody fragments, antigenic fragments of BRHF1 polypeptides, or BRHF1 nucleic acids of the invention.

The nucleic acids of the present invention can be utilized as probes or primers to detect BRHF1 nucleic acids. For example, a polynucleotide probe or primer comprising a polynucleotide selected from the group consisting of at least 10 contiguous nucleotides of SEQ ID NO: 3, at least 10 contiguous nucleotides of SEQ ID NO: 4, at least 10 contiguous nucleotides from SEQ ID NO: 5 and at least 10 contiguous nucleotides of SEQ ID NO: 9 can be utilized to detect a BRHF1 nucleic acid. The probes or primers generated from SEQ ID NO: 5 can be utilized to detect the genomic insertion on chromosome 17 associated with cancer. Therefore, the polynucleotide probes or primers of this invention can be at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or at least 200 nucleotides in length.

Further provided by the present invention is a method for detecting the presence of cancer in a subject, comprising: (a) contacting a biological sample obtained from a subject with at least two oligonucleotide primers, each primer consisting of 10 to 200 contiguous nucleotides of SEQ ID NO: 5 or the complement thereof, in a reverse transcriptase polymerase chain reaction; and (b) detecting in the sample a polynucleotide sequence that amplifies in the presence of said oligonucleotide primers, wherein the presence of an amplified polynucleotide sequence indicates the presence of cancer in the subject.

For example, one of skill in the art could contact a biological sample with a primer comprising nucleotides 1-10 of SEQ ID NO: 5 or a complement thereof and a primer comprising nucleotides 1253 to 1263 of SEQ ID NO: 5 or a complement thereof in order to detect the presence of cancer in a subject. One of skill in the art could also contact a biological sample with a primer comprising nucleotides 1-20 of SEQ ID NO: 5 or a complement thereof and a primer comprising nucleotides 1243 to 1263 of SEQ ID NO: 5 or a complement thereof in order to detect the presence of cancer in a subject. One skilled in the art could also contact a biological sample with a primer comprising nucleotides 5-25 of SEQ ID NO: 5 or a complement thereof and a primer comprising nucleotides 1253 to 1263 of SEQ ID NO: 5 or a complement thereof in order to detect the presence of cancer in a subject. These examples are merely exemplary of the numerous combinations of primers that can be utilized by one of skill in the art to detect the presence of cancer in a subject by the methods described herein.

Also provided by the present invention is a method for detecting the presence of cancer in a subject, comprising: (a) contacting a biological sample obtained from a subject with a nucleic acid probe under conditions that allow the probe to selectively bind a BRHF1 nucleic acid; and (b) detecting the presence of a BRHF1 nucleic acid, whereby the presence of a BRHF1 nucleic acid indicates the presence of cancer in the subject.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein. One of skill in the art would know how to design a probe that specifically hybridizes to a BRHF1 nucleic acid and not to a non-BRHF1 nucleic acid. The probes and primers of the present invention can also be unique BRHF1 nucleic acids. One of skill in the art can also analyze the nucleotide sequence of BRHF1 and select regions that are unique to BRHF1 to design the primers and probes of the present invention. To be unique, the BRHF1 nucleotide sequence must be of sufficient size to distinguish it from other known sequences, most readily determined by comparing any nucleic acid fragment to the nucleotide sequences of nucleic acids in computer databases, such as GenBank. Such comparative searches are standard in the art.

Stringent conditions refers to the washing conditions used in a hybridization protocol. In general, the washing conditions should be a combination of temperature and salt concentration chosen so that the denaturation temperature is approximately 5-20° C. below the calculated Tₘ of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C.

The following is an exemplary set of hybridization conditions and is not limiting:

Very High Stringency

| | |
|---|---|
| Hybridization: | 5× SSC at 65° C. for 16 hours |
| Wash twice: | 2× SSC at room temperature (RT) for 15 minutes |
| | each |
| Wash twice: | 0.5× SSC at 65° C. for 20 minutes each |

High Stringency

| | |
|---|---|
| Hybridization: | 5×-6× SSC at 65° C.-70° C. for 16-20 hours |
| Wash twice: | 2× SSC at RT for 5-20 minutes each |
| Wash twice: | 1× SSC at 55° C.-70° C. for 30 minutes each |

Low Stringency

| | |
|---|---|
| Hybridization: | 6× SSC at RT to 55° C. for 16-20 hours |
| Wash at least | 2×-3× SSC at RT to 55° C. for 20-30 minutes each. |
| twice: | |

A number of methods are available for analyzing nucleic acids for the presence of a specific sequence. For all of the methods described herein, genomic DNA can be extracted from a sample and this sample can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. Such samples can be obtained directly from a subject, commercially obtained or obtained via other means. Thus, the invention described herein can be utilized to analyze a nucleic acid sample that comprises genomic DNA, amplified DNA (such as a PCR product) cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired methods, which are known to the skilled artisan, such as DNase treatment or shearing of the genomic DNA, preferably lightly.

If sufficient DNA is available, genomic DNA can be used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify SEQ ID NO: 5 or a fragment thereof. For example, the present invention provides forward primer (5′-CAGAGCCTGT-3′) (SEQ ID NO: 6) and reverse primer (5′-CTCTGGGACA-3′) (SEQ ID NO: 7) which can be utilized to amplify the genomic region of chromosome 17 comprising an insertion of approximately 1270 base pairs associated with cancer.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Since applicants have provided the genomic locus for an insertion on chromosome 17q11.2, one of skill in the art can analyze genomic DNA either directly or after amplification for the presence of this insertion by sequencing this region of chromosome 17q11.2 and identifying the insertion at this genomic location.

One of skill in the art can also utilize FISH with probes localized to chromosome 17 or probes localized to region 17q11.2 to identify the insertion (See Hemel and Eussen “Interchromosomal insertions. Identification of five cases and a review” Hum. Genet. 107(5):415-32 (2000); Engelen et al. “Duplication within chromosome region 15q11-q13 in a patient with similarities to Prader-Willi syndrome confirmed by region-specific and band specific fish” Genet. Couns. 10(2):123-32 (1999); Lorda-Sanchez et al. “Proximal partial 5p trisomy resulting from a maternal (19; 5) insertion” Am. J. Med. Genet. 68(4):476-80 (1997) All of these references are incorporated herein by this reference as they pertain to FISH analysis and techniques utilized FISH to identify chromosomal aberrations, such as insertions, deletions and duplications).

The sample nucleic acid, e.g. amplified or cloned fragment, can be analyzed by one of a number of methods known in the art. The nucleic acid can be sequenced by dideoxy or other methods. Hybridization with the sequence can also be used to determine its presence, by Southern blots, dot blots, etc.

The BRHF1 nucleic acids of the invention can also be used in polynucleotide arrays. Polynucleotide arrays provide a high throughput technique that can assay a large number of polynucleotide sequences in a single sample. This technology can be used, for example, as a diagnostic tool to identify metastatic lesions or to assess the metastatic potential of a tumor.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NOS: 3, 4, 5 and 9.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip.™, can also be used. Use of the GeneChip.™. to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Tissue samples which are suspected of being metastatic or the metastatic potential of which is unknown can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels which can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance.

Further provided by the present invention is a method of determining the stage of cancer in a subject comprising: (a) contacting a biological sample obtained from a subject with a nucleic acid probe under conditions that allow the probe to selectively bind a BRHF1 nucleic acid, (b) detecting the presence of a BRHF1 nucleic acid and correlating the amount of BRHF1 nucleic acid in the sample with a clinically defined stage of cancer, thereby determining the stage of cancer in the subject.

Since therapy and clinical decisions are often dependent on diagnosis and staging, BRHF1 detection with BRHF1 probes allows correlation of BRHF1 nucleic acid levels with a particular stage of cancer. One skilled in the art would be able to measure BRHF1 nucleic acid levels (for example, mRNA levels) in numerous subjects in order to establish ranges of BRHF1 levels that correspond to clinically defined stages of cancer. The process of determining the clinical stages of cancer would be known to one of skill in the art. These ranges will allow the skilled practitioner to measure BRHF1 nucleic acid levels in a subject diagnosed with cancer and correlate the levels in each subject with a range that corresponds to a stage of cancer. Any of the detection methods described herein can be utilized with other detection methods, including, but not limited to, visual inspection, histopathological examination, radiological examination, biopsy and MRI to confirm the presence of cancer, to confirm the type of cancer and/or to determine a clinical stage of cancer.

A person skilled in the art would know that the methods of this invention can also be utilized to test the efficacy of an anti-cancer treatment. For example, if the patient diagnosed with cancer is assayed for BRHF1 nucleic acid levels prior to the administration of an anti-cancer treatment and assayed at a second time point after the administration of the anti-cancer treatment, a decrease in BRHF1 nucleic acid levels may indicate an effective anti-cancer treatment had been administered. The skilled practitioner will associate the decreases observed with a particular level of effectiveness. If no decrease is observed, the anti-cancer treatment may need to be adjusted.

Furthermore, since the increased expression of BRHF1 is associated with estrogen dependent breast cancers, upon detection of increased levels of BRHF1 or correlation of BRHF1 levels with a particular stage of breast cancer, the skilled practitioner can administer a therapy suited for the treatment of estrogen-dependent breast cancer.

The patients of this invention undergoing anti-cancer therapy can include patients undergoing surgery, chemotherapy, radiotherapy, immunotherapy or any combination thereof. Examples of chemotherapeutic agents include cisplatin, 5-fluorouracil and S-1. Immunotherapeutics methods include administration of interleukin-2 and interferon-α.

Antibody Detection Methods

The present invention provides a method of detecting the presence of cancer in a subject comprising: a) contacting a sample from the subject with an antibody to a BRHF1 polypeptide; and b) detecting the antibody bound to the BRHF1 polypeptide in the sample, wherein binding of BRHF1 polypeptide to the antibody indicates the presence of a BRHF1 polypeptide in the sample, the presence of a BRHF1 polypeptide indicating the presence of cancer in the subject.

Any of the detection methods described herein can be utilized to detect any BRHF1 polypeptide and can be utilized for initial diagnosis of cancer as well as in methods of determining the progression of cancer in a subject or determining the efficacy of treatment. The antibody, or fragment thereof, can be linked to a detectable label either directly or indirectly through use of a secondary and/or tertiary antibody; thus, bound antibody, fragment or molecular complex can be detected directly in an ELISA or similar assay.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of a BRHF1 polypeptide can be on, supported by, or attached to a substrate which facilitates detection. Such a substrate can be a mobile solid support. Thus, provided by the invention are substrates including one or more of the antibodies or antibody fragments, or antigenic fragments of a BRHF1 polypeptide.

Further provided by the present invention is a method of determining the stage cancer in a subject comprising: a) contacting a sample from the subject with an antibody to a BRHF1 polypeptide, b) detecting the antibody bound to the BRHF1 polypeptide in the sample, and correlating the amount of bound antibody in the sample with a clinically defined stage of cancer, thereby determining the stage of cancer in the subject.

Since therapy and clinical decisions are often dependent on diagnosis, BRHF1 detection with this antibody or fragment thereof allows correlation of BRHF1 polypeptide levels with a particular stage of cancer. One skilled in the art would be able to measure BRHF1 levels in numerous subjects in order to establish ranges of BRHF1 levels that correspond to clinically defined stages of cancer. The process of determining the clinical stages of cancer would be known to one of skill in the art. These ranges will allow the skilled practitioner to measure BRHF1 levels in a subject diagnosed with cancer and correlate the levels in each subject with a range that corresponds to a stage of cancer.

A person skilled in the art would know that the methods of this invention can also be utilized to test the efficacy of an anti-cancer treatment. For example, if the patient diagnosed with cancer is assayed for BRHF1 levels prior to the administration of an anti-cancer treatment and assayed at a second time point after the administration of the anti-cancer treatment, a decrease in BRHF1 levels may indicate an effective anti-cancer treatment had been administered. The skilled practitioner will associate the decreases observed with a particular level of effectiveness. If no decrease is observed, the anti-cancer treatment may need to be adjusted.

Furthermore, since the increased expression of BRHF1 is associated with estrogen dependent breast cancers, upon detection of increased levels of BRHF1 or correlation of BRHF1 levels with a particular stage of breast cancer, the skilled practitioner can administer a therapy suited for the treatment of estrogen-dependent breast cancer.

The patients of this invention undergoing anti-cancer therapy can include patients undergoing surgery, chemotherapy, radiotherapy, immunotherapy or any combination thereof. Examples of chemotherapeutic agents include cisplatin, 5-fluorouracil and S-1. Immunotherapeutics methods include administration of interleukin-2 and interferon-α.

Inhibition of BRHF1

The present invention provides a method of reducing BRHF1 expression in a cell comprising administering to the cell an antisense oligonucleotide that specifically binds to mRNA transcribed from the BRHF1 gene under conditions that allow hybridization, wherein the BRHF1 mRNA comprises a nucleotide sequence selected from the group consisting of at least 15 contiguous nucleotides from SEQ ID NO: 3, at least 15 contiguous nucleotides from SEQ ID NO 4 and at least 15 contiguous nucleotides from SEQ ID NO: 9, and wherein hybridization of the antisense oligonucleotide with the BRHF1 mRNA reduces BRHF1 expression. For example, a sense strand sequence derived from SEQ ID NO: 3 (5′tgagcacaactattccgatc3′) (SEQ ID NO: 10) can be utilized to generate BRHF-1 specific antisense oligonucleotide. This antisense oligonucleotide can comprise (actcgtgttgataaggctag) (SEQ ID NO: 15). As another example, a sense strand sequence derived from SEQ ID NO: 4 (5′aagcaacttcagcaaagtctcag 3′) (SEQ ID NO: 11) can be utilized to generate BRHF-1 specific antisense oligonucleotide. This antisense oligonucleotide can comprise (ttcgttgaagtcgtttcagagtc) (SEQ ID NO: 16). Other examples of antisense molecules that can be utilized include, but are not limited to nucleic acids comprising (ctcgtgttgataaggctagt) (SEQ ID NO: 17), (tcgtgttgataaggctagtc) (SEQ ID NO: 18) (cgtgttgataaggctagtcg) (SEQ ID NO: 19), (tcgttgaagtcgtttcagagtcc) (SEQ ID NO: 20) or (gttgaagtcgtttcagagtcct) (SEQ ID NO: 21).

As used herein, “reducing” or “inhibiting” means a decrease in a BRHF1 expression levels. Such reduction does not have to be complete and can range from a slight decrease in BRHF1 expression levels to complete inhibition of BRHF1 expression levels.

Antisense technology is well known in the art and describes a mechanism whereby a nucleic acid comprising a nucleotide sequence which is in a complementary, “antisense” orientation with respect to a coding or “sense” sequence of an endogenous gene, is introduced into a cell, whereby a duplex may form between the antisense sequence and its complementary sense sequence. The formation of this duplex may result in inactivation of the endogenous gene.

For example, the antisense nucleic acid can inhibit gene expression by forming an RNA/RNA duplex between the antisense RNA and the RNA transcribed from a target gene. The precise mechanism by which this duplex formation decreases the production of the protein encoded by the endogenous gene most likely involves binding of complementary regions of the normal sense mRNA and the antisense RNA strand with duplex formation in a manner that blocks RNA processing and translation. Alternative mechanisms include the formation of a triplex between the antisense RNA and duplex DNA or the formation of a DNA-RNA duplex with subsequent degradation of DNA-RNA hybrids by RNAse H. Furthermore, an antisense effect can result from certain DNA-based oligonucleotides via triple-helix formation between the oligomer and double-stranded DNA which results in the repression of gene transcription. Antisense nucleic acid can be produced for any relevant endogenous gene for which the coding sequence has been or can be determined according to well known methods.

A nucleic acid encoding an antisense RNA can be selected based on the protein desired to be inhibited or decreased in cells, by providing an RNA that will selectively bind to the cellular mRNA encoding such protein. Binding of the antisense molecule to the target mRNA may incapacitate the mRNAs, thus preventing its translation into a functional protein. The antisense RNA/mRNA complexes can then become a target for RNAse-H and are eventually degraded by the host cell RNAse-H. Control regions, such as enhancers and promoters, can be selected for antisense RNA targeting according to the cell or tissue in which it is to be expressed, as is known in the art. Preferable antisense-encoding constructs can encode full-length complements to target sequences; however, smaller length sequences down to oligonucleotide size can be utilized. For example, the antisense-encoding constructs can encode fill-length complements to the BRHF1 gene, smaller length sequences or oligonucleotide sequences.

The present invention also provides a method of reducing BRHF1 expression comprising administering to a cell a ribozyme that specifically binds to mRNA transcribed from the BRHF1 gene, the ribozyme binding reducing BRHF1 expression.

Therefore, when BRHF1 expression can be decreased using a ribozyme, an RNA molecule with catalytic activity. See, e.g., Cech, 1987, Science 236: 1532-1539; Cech, 1990, Ann. Rev. Biochem. 59:543-568; Cech, 1992, Curr. Opin. Struct. Biol. 2: 605-609; Couture and Stinchcomb, 1996, Trends Genet. 12: 510-515. Ribozymes can be used to inhibit gene function by cleaving an RNA sequence, as is known in the art (e.g., Haseloff et al., U.S. Pat. No. 5,641,673).

The coding sequence of BRHF1 can be used to generate a ribozyme which will specifically bind to mRNA transcribed from a BRHF1 gene. Methods of designing and constructing ribozymes which can cleave other RNA molecules in trans in a highly sequence specific manner have been developed and described in the art (see Haseloff et al. (1988), Nature 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Ribozymes can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells.

Mechanical methods, such as microinjection, liposome-mediated transfection, electroporation, or calcium phosphate precipitation, can be used to introduce the ribozyme-containing DNA construct into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

As taught in Haseloff et al., U.S. Pat. No. 5,641,673, the ribozyme can be engineered so that its expression will occur in response to factors which induce expression of BRHF1. The ribozyme can also be engineered to provide an additional level of regulation, so that destruction of mRNA occurs only when both the ribozyme and the BRHF1 gene are induced in the cells.

Further provided by the present invention is a method of reducing BRHF1 expression comprising administering to a cell an siRNA that is complementary to at least a portion of the coding sequence of BRHF1, under conditions that allow hybridization of the siRNA with the BRHF1 coding sequence, wherein the BRHF1 coding sequence comprises a nucleotide sequence selected from the group consisting of at least 21 contiguous nucleotides of SEQ ID NO: 3 and at least 21 contiguous nucleotides of SEQ ID NO: 4 and wherein the binding of the siRNA to the BRHF1 coding sequence reduces BRHF1 expression. For example, a sense strand sequence derived from SEQ ID NO:4 (5′AAACCACTGCTCAACGAAATA3′) (SEQ ID NO: 12) can be utilized to generate a siRNA oligonucleotide template. As another example, a sense strand sequence derived from SEQ ID NO:3 (5′AAGGGATTATCGCCTATCGCC3′) (SEQ ID NO: 13) can be utilized to generate a siRNA oligonucleotide template.

Also provided by the present invention is a method of reducing the activity of a BRHF1 polypeptide, comprising contacting the protein or the cell with one or more anti-BRHF1 antibodies of the present invention. Activities of BRHF1 include, but are not limited to, BRHF1 binding to a cell, BRHF1 binding to another polypeptide, BRHF1 binding to a nucleic acid or BRHF1 binding to a receptor. Other activities of BRHF1 include promoting increased cell proliferation and other activities associated with cancer. As used herein, “reducing” means a decrease in a BRHF1 activity as compared to a control. The control can include BRHF1 polypeptides or a cell expressing a BRHF1 polypeptide in the absence of the antibody or other agent. Thus, the control can include a cell or protein before or after contact with the antibody or other agent or can include a cell or protein that is not contacted with the antibody or agent. Such a reduction in activity does not have to be complete and can range from a slight decrease in BRHF1 activity to complete inhibition of BRHF1 activity.

Methods of inhibiting or reducing BRHF1 expression as well as methods of inhibiting or reducing BRHF1 activity can be utilized to treat subjects with cancer or other proliferative disorders. As used herein, “treating” describes an improvement in the patient's clinical state. The improvement may range from reduction of the symptoms of the disease to complete amelioration of the disease.

Optimal dosages used will vary according to the individual being treated and the inhibitor being used. The amount of inhibitor will also vary among individuals on the basis of age, size, weight, condition, etc. One skilled in the art will realize that dosages are best optimized by the practicing physician and methods for determining dose amounts and regimens and preparing dosage forms are described, for example, in Remington's Pharmaceutical Sciences. For example, suitable doses and dosage regimens can be determined by comparison to agents presently used in the treatment or prevention of inflammation or autoimmune disorders.

Typically, the inhibitor of this invention can be administered orally or parenterally in a dosage range of 0.1 to 100 mg/kg of body weight depending on the clinical response that is to be obtained. Administration of inhibitor can be stopped completely following a prolonged remission or stabilization of disease signs and symptoms and readministered following a worsening of either the signs or symptoms of the disease.

The efficacy of administration of a particular dose of inhibitor in treating cancer as described herein can be determined by evaluating the particular aspects of the medical history, the signs, symptoms and objective laboratory tests that have a documented utility in evaluating pathophysiological activity of the particular cancer being treated. These signs, symptoms and objective laboratory tests will vary depending on the particular disorder being treated, as will be well known to any clinician in this field. For example, if the progression of cancer is shown to be reduced or ameliorated then a particular treatment can be considered efficacious.

Once it is established that disease activity is significantly improved or stabilized by a particular inhibitor, specific signs, symptoms and laboratory tests can be evaluated in accordance with a reduced or discontinued treatment schedule. If a disease activity recurs, based on standard methods of evaluation of the particular signs, symptoms and objective laboratory tests as described herein, treatment can be reinitiated.

The present invention also provides inhibitors of BRHF1 expression and/or activity, in a pharmaceutically acceptable carrier. By “pharmaceutically acceptable” is meant a material that is not biologically or otherwise undesirable, i.e., the material may be administered to an individual along with the selected compound without causing any undesirable biological effects or interacting in a undesirable manner with any of the other components of the pharmaceutical composition in which it is contained. The carrier may depend on the method of administration and the particular patient. It is also noted that not all methods of administering the inhibitors of BRHF1 described herein require a pharmaceutically acceptable carrier.

In the present invention, the inhibitors can be orally or parenterally administered in a carrier pharmaceutically acceptable to human subjects. Suitable carriers for oral or inhaled administration can include one or more of the carriers pharmaceutically acceptable to human subjects. Suitable carriers for oral administration include one or more substances which may also act as a flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pyrogen free saline, pharmaceutically accepted oils, or a mixture of any of these. The liquid can also contain other suitable pharmaceutical addition such as buffers, preservatives, flavoring agents, viscosity or osmo-regulators, stabilizers or suspending agents. Examples of suitable liquid carriers include water with or without various additives, including carboxypolymethylene as a ph-regulated gel. The inhibitor may be contained in enteric coated capsules that release the polypeptide into the intestine to avoid gastric breakdown. For parenteral administration of the antagonist, a sterile solution or suspension is prepared in saline that may contain additives, such as ethyl oleate or isopropyl myristate, and can be injected for example, into subcutaneous or intramuscular tissues, as well as intravenously.

Similarly, a nucleic acid such as an antisense nucleic acid, a ribozyme, an siRNA or a nucleic encoding a polypeptide inhibitor of BRHF1 can be administered. The nucleic acids of this invention can be delivered to the cells of a subject either in vivo or ex vivo by standard methods. In the methods described herein which include the introduction of exogenous DNA into the cells of a subject (i.e., gene transduction or transfection), the nucleic acids of the present invention can be in the form of naked DNA or the nucleic acids can be in a vector for delivering the nucleic acids to the cells. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as Lipofectin®, Lipofectamine® (GIBCO-BRL, Inc., Gaithersburg, Md.), Superfect® (Qiagen, Inc. Hilden, Germany) and Transfectam® (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a Sonoporation machine (ImaRx Pharmaceutical Corp., Tucson, Ariz.).

Other vectors than can be utilized for delivery include, but are not limited to adeno-associated viral (AAV) vectors, recombinant herpes virus (HSV), lentiviral vectors, pseudotyped retroviral vectors, and pox virus vectors, such as vaccinia virus vectors. Physical transduction techniques can also be used, such as liposome delivery and receptor-mediated and other endocytosis mechanism. This invention can be used in conjunction with any of these or other commonly used gene transfer methods.

For in vivo administration, the cells can be in a subject and the nucleic acid can be administered in a pharmaceutically acceptable carrier. The subject can be any animal in which it is desirable to deliver nucleic acid of the present invention in a cell. In a preferred embodiment, the subject of the present invention is a human. In addition, non-human subjects include, but are not limited to, cats, dogs, birds, horses, cows, goats, sheep, guinea pigs, hamsters, gerbils and rabbits.

Screening Methods

The present invention provides a method of identifying a compound that reduces BRHF1 expression, comprising administering a test compound to a cell containing a BRHF1 gene and detecting the level of the BRHF1 gene product produced, a decrease in the gene product as compared to a control cell indicating the compound reduces BRHF1 expression.

The “cells” utilized in the screening methods of this invention include any cell type, cancerous or noncancerous, that may express or be affected by the expression of a BRHF1 gene or the activity of a BRHF1 protein. Examples include, but are not limited to, breast cancer cells, colorectal cancer cells, pancreatic cancer cells, gastrointestinal cancer cells and prostate cancer cells. The cell could also include a cell that comprises an exogenous nucleic acid that encodes BRHF1.

As utilized herein, “a BRHF1 gene product” can be, for example BRHF1 mRNA, or a BRHF1 polypeptide. Methods of detecting a BRHF1 nucleic acid and a BRHF1 polypeptide are discussed herein. Furthermore, these methods are not limited to those discussed herein as there are numerous methods for detecting and measuring expression known to those skilled in the art.

Further provided by this invention is a method of identifying a compound that reduces BRHF1 expression, comprising administering a test compound to a cell containing a nucleic acid encoding SEQ ID NO: 1 and detecting the level of the BRHF1 gene product produced, a decrease in the gene product indicating a compound that reduces BRHF1 expression.

Also provided is a method of identifying a compound that reduces BRHF1 expression in the presence of estrogen, comprising administering a test compound and estrogen to a cell containing a BRHF1 gene and detecting the level of the BRHF1 gene product produced, a decrease in the gene product as compared to a control cell indicating a compound that reduces BRHF1 expression in the presence of estrogen.

The present invention also provides a method of identifying a compound that reduces BRHF1 expression in the presence of estrogen, comprising administering a test compound and estrogen to a cell containing a nucleic acid encoding the polypeptide of claim 1 and detecting the level of the BRHF1 gene product produced, a decrease in the gene product indicating a compound that reduces BRHF1 expression in the presence of estrogen.

### EXAMPLES

A Novel BRHF1 Gene

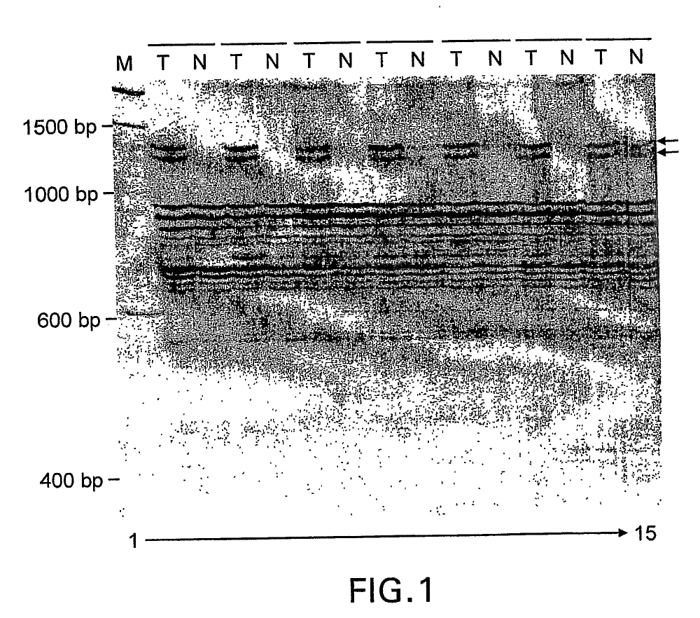

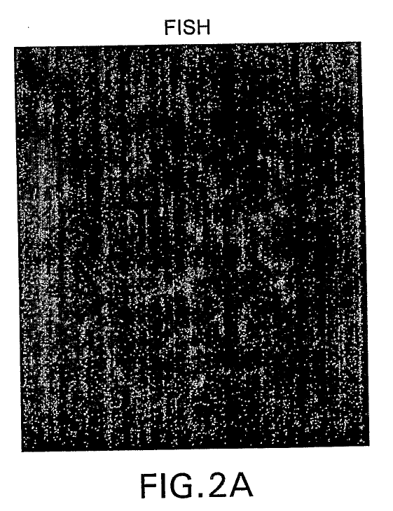

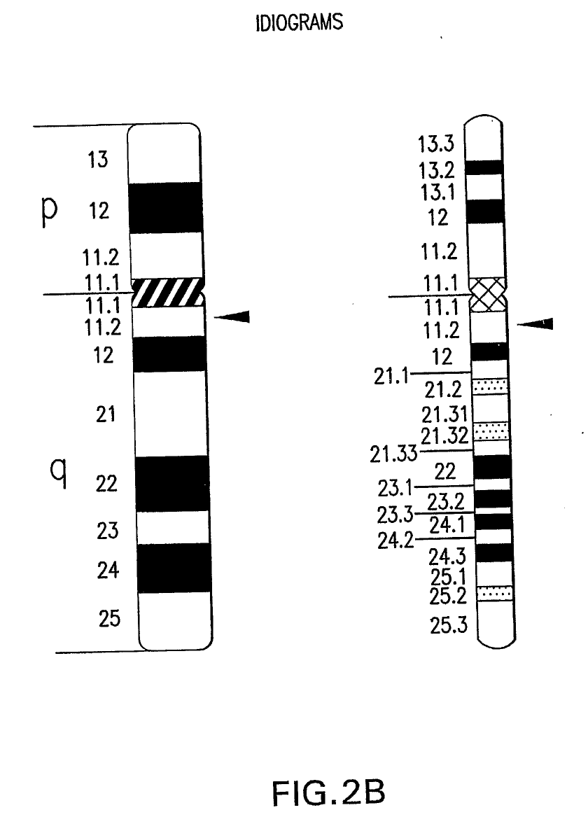

The present invention shows that BRHF1 gene is mutated with very high frequency (81% of the samples analyzed) in human breast tumors: Using Arbitrary-Primed PCR (AP-PCR) mutations in the human breast cancer genome were found. An insertion of an approximately 1270 bp amplification product (resolved on agarose gel) was observed in 81% (17 out of 21) of the tumor samples as compared with the uninvolved breast tissue from the same patient. The 1270 bp amplification product resolved into two bands of 1200 and 1300 bp when applied on the denaturing sequencing gel (FIG. 1).

To confirm the purity (i.e., origin from a single genomic region) of this amplification product, the agarose gel eluted fragment was cloned, and then run on a sequencing gel, where it again resolved into two bands (1200 and 1300 bp), indicating that the two bands represent the two single-stranded fragments of the 1270 bp double-stranded DNA fragment. This suggests a semi-stable conformation in this DNA region.

BRHF1 is Located on q^{11.2} Region of Chromosome 17, a Highly Unstable Chromosome in Breast Cancer

Plasmid DNA from BAC clone HCIT 54B22 (Research Genetics, Huntsville, Ala.) that contains a BRHF-1 gene sequence was labeled with digoxigenin dUTP by nick translation. A specific hybridization signal was detected by incubating the hybridized slides in fluorescent antidigoxigenin antibodies followed by counterstaining with DAPI. Measurements in 10 cells showing a specifically labeled chromosome 17 demonstrated that the BRHF1 gene is located at a position that corresponds to 17q^{11.2}.

BRHF1 has Significant Sequence Homology with a Known Uncharacterized cDNA Clone Reported from Human Breast Tumor

The 1270 bp AP-PCR amplification product from the breast tumor DNA was cloned into a PCRII vector using TA cloning kit (Invitrogen, CA). Ten clones were sequenced to confirm the insert sequence, and the final sequence was compared with known sequences in the Gen Bank database using the BLAST program. BLAST analysis revealed 99% homology of this fragment with one of the genomic clones (HCIT 542B22, Accession number AC004253). The homology search using dbEST revealed that the tumor-specific 1270 bp amplified fragment had 93% similarity with one of the human EST clone (Accession # BE 065399) sequence. Interestingly, this EST has been reported recently from cDNA derived from human breast tumor tissue (Dias et al. 2000). ORF analysis predicted an ORF of 270 amino acid within this sequence. RT-PCR analysis confirmed that this ORF is transcriptionally active. Sequence analysis revealed point mutations in tumors at the primer binding site, and these mutations resulted in amino acid substitutions (Isoleucine [in normal]→Valine [in tumor]; Aspartic acid [in normal]→Arginine [in tumor]) in the predicted ORF.

The BRHF1 Gene Transcript Size is 6.50 kb and is Specific to Estrogen Dependent Breast Cancer

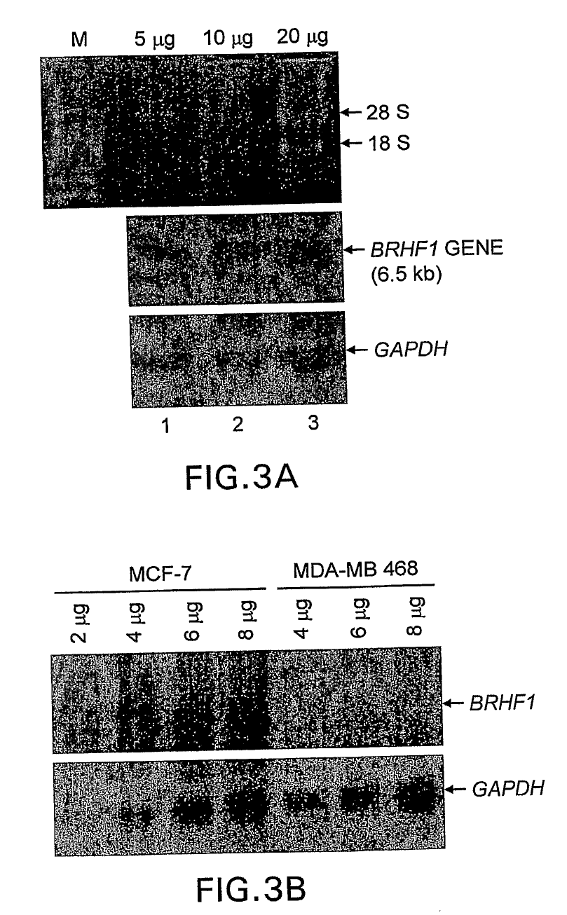

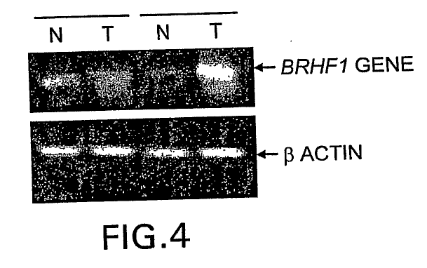

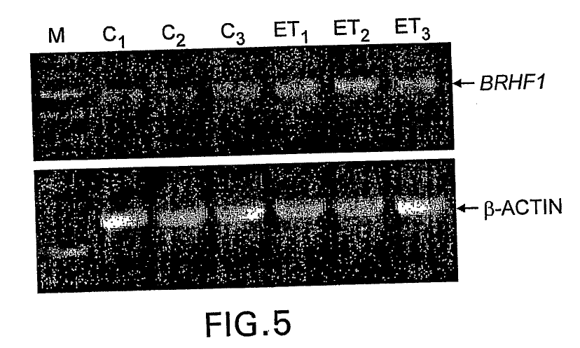

Northern blot analysis of the RNA isolated from the MCF7 human breast cancer cell line revealed a 6.5 kb transcript of the BRHF1 gene (FIG. 3a). The probe used for the Northern blot analysis was PCR amplified from the cDNA using the primer set specific for the ORF region of the BRHF1 gene. To ensure the specificity of the probe DNA, the PCR product was cloned and sequenced. Our preliminary data of Northern blot analysis with RNA from a ER-positive breast cancer cell line (MCF7) and a ER-negative breast cancer cell line (MDA-MB 468) indicate that this gene is differentially expressed in MCF7 and not in ER-negative MDA-MB 468 breast cancer cells (FIG. 3b).

BRHF1 Transcript is Multifold Higher in Breast Tumors as Compared to the Matched Uninvolved Normal Breast Tissue

Primers were designed from the ORF region of the BRHF1 gene sequence. Using the TRIzol method (Invitrogen), total RNA was isolated from tumors and from corresponding uninvolved normal breast tissues. cDNA was synthesized using the first strand cDNA synthesis kit (Invitrogen). RT-PCR analysis revealed multifold increased expression of this gene in breast tumors as compared to uninvolved normal tissue.

BRHF1 Gene Transcript is Upregulated by Estrogen Treatment

Higher level of gene transcript level measured by RT-PCR from estrogen (17 β-estradiol) treated-breast cancer cells as compared with the untreated control cells indicate that BRHF1 gene is estrogen responsive (FIG. 5).

In summary, the present invention provides mutations in an uncharacterized region of the breast cancer genome located at 17q11.2. This uncharacterized genomic region harboring mutation represents a part of a novel gene (BRHF1) and this BRHF1 gene harbors mutations in 81% of the sporadic breast tumor samples analyzed. The full-length cDNA sequence of this gene is 6.5 kb. These studies also showed that the BRHF1 gene is upregulated in human breast tumors as the BRHF1 gene is expressed only in the ER-positive breast cancer cells, not in ER-negative breast cancer cells. Furthermore, this invention showed that BRHF1 is an estrogen-responsive gene in breast cancer.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A purified polypeptide comprising SEQ ID NO: 1.

2. A purified polypeptide comprising an amino acid sequence at least about 95% identical to the sequence of SEQ ID NO: 1.

3. A purified polypeptide comprising the amino acid sequence of SEQ ID NO: 1 with one or more conservative amino acid substitutions.

4. A nucleic acid encoding the polypeptide of claim 1.

5. The nucleic acid of claim 4, wherein the nucleic acid comprises SEQ ID NO: 3

6. A vector comprising the nucleic acid of claim 4 or 5.

7. A host cell comprising the vector of claim 6.

8. The host cell of claim 7, wherein the host cell is a prokaryotic cell.

9. The host cell of claim 8, wherein the prokaryotic cell is an E. coli cell.

10. The host cell of claim 7, wherein the host cell is a eukaryotic cell.

11. A purified polypeptide comprising SEQ ID NO: 2.

12. A purified polypeptide comprising an amino acid sequence at least about 95% identical to the sequence of SEQ ID NO:2.

13. A purified polypeptide comprising the amino acid sequence of SEQ ID NO:2 with one or more conservative amino acid substitutions.

14. A nucleic acid encoding the polypeptide of claim 11.

15. The nucleic acid of claim 14, wherein the nucleic acid comprises SEQ ID NO: 4

16. A vector comprising the nucleic acid of claim 14 or 15.

17. A host cell comprising the vector of claim 16.

18. The host cell of claim 17, wherein the host cell is a prokaryotic cell.

19. The host cell of claim 18, wherein the prokaryotic cell is an E. coli cell.

20. The host cell of claim 17, wherein the host cell is a eukaryotic cell.

21. A purified polypeptide comprising SEQ ID NO: 8.

22. A purified polypeptide comprising an amino acid sequence at least about 95% identical to the sequence of SEQ ID NO:8.

23. A purified polypeptide comprising the amino acid sequence of SEQ ID NO:8 with one or more conservative amino acid substitutions.

24. A nucleic acid encoding the polypeptide of claim 21.

25. The nucleic acid of claim 24, wherein the nucleic acid comprises SEQ ID NO: 9

26. A vector comprising the nucleic acid of claim 24 or 25.

27. A host cell comprising the vector of claim 26.

28. The host cell of claim 27, wherein the host cell is a prokaryotic cell.

29. The host cell of claim 28, wherein the prokaryotic cell is an E. coli cell.

30. The host cell of claim 27, wherein the host cell is a eukaryotic cell.

31. An isolated antibody or fragment thereof that specifically binds the polypeptide of claim 21.

32. The antibody of claim 31, wherein the antibody is a polyclonal antibody.

33. The antibody of claim 31, wherein the antibody is a monoclonal antibody.

34. The antibody of claim 31, wherein the antibody is labeled with a detectable moiety.

35. The antibody of claim 34, wherein the detectable moiety is selected from the group consisting of a fluorescent moiety, an enzyme-linked moiety, a biotinylated moiety and a radiolabeled moiety.

36. The antibody of claim 31, wherein the antibody is humanized.

37. An isolated antibody or fragment thereof that specifically binds the polypeptide of claim 11.

38. The antibody of claim 37, wherein the antibody is a polyclonal antibody.

39. The antibody of claim 37, wherein the antibody is a monoclonal antibody.

40. The antibody of claim 37, wherein the antibody is labeled with a detectable moiety.

41. The antibody of claim 40, wherein the detectable moiety is selected from the group consisting of a fluorescent moiety, an enzyme-linked moiety, a biotinylated moiety and a radiolabeled moiety.

42. The antibody of claim 37, wherein the antibody is humanized.

43. A method for detecting the presence of cancer in a subject, comprising the steps of:
(a) contacting a biological sample obtained from a subject with at least two oligonucleotide primers, each primer consisting of 10 to 200 contiguous nucleotides of SEQ ID NO: 5 or the complement thereof, in a reverse transcriptase polymerase chain reaction; and
(b) detecting in the sample a polynucleotide sequence that amplifies in the presence of said oligonucleotide primers, wherein the presence of an amplified polynucleotide sequence indicates the presence of cancer in the subject.

44. The method of claim 43, wherein the cancer is breast cancer.

45. The method of claim 34, wherein the breast cancer is estrogen dependent breast cancer.

46. The method of claim 33, wherein the primers are forward primer (5′-CAGAGCCTGT-3′) (SEQ ID NO: 6) and reverse primer (5′-CTCTGGGACA-3′) (SEQ ID NO: 7).

47. A polynucleotide probe comprising a polynucleotide selected from the group consisting of at least 25 contiguous nucleotides of SEQ ID NO: 3, at least 25 contiguous nucleotides of SEQ ID NO: 4, at least 25 contiguous nucleotides of SEQ ID NO: 5 and at least 25 contiguous nucleotides of SEQ ID NO: 9.

48. A method for detecting the presence of cancer in a subject, comprising the steps of:
(a) contacting a biological sample obtained from a subject with the probe of claim 47 under conditions that allow the probe to selectively bind a BRHF1 nucleic acid; and
(b) detecting the presence of a BRHF1 nucleic acid, whereby the presence of a BRHF1 nucleic acid indicates the presence of cancer in the subject.

49. The method of claim 48, wherein the cancer is breast cancer.

50. The method of claim 49, wherein the breast cancer is estrogen dependent breast cancer.

51. The method of claim 48, further comprising:
d) measuring the amount of BRHF1 nucleic acid in the sample and correlating this amount with a particular stage of cancer.

52. A method of detecting the presence of cancer in a subject comprising:
a) contacting a sample from the subject with an antibody to a BRHF1 polypeptide; and
b) detecting the antibody bound to the BRHF1 polypeptide in the sample, wherein binding of BRHF1 polypeptide to the antibody indicates the presence of a BRHF1 polypeptide in the sample, the presence of a BRHF1 polypeptide indicating the presence of cancer in the subject.

53. The method of claim 52, wherein the cancer is breast cancer.

54. The method of claim 53, wherein the breast cancer is estrogen dependent breast cancer.

55. The method of claim 52 further comprising:
d) measuring the amount of BRHF1 polypeptide in the sample and correlating this amount with a particular stage of cancer.

56. A method of reducing BRHF1 expression in a cell comprising administering to the cell an antisense oligonucleotide that specifically binds to mRNA transcribed from the BRHF1 gene under conditions that allow hybridization, wherein the BRHF1 mRNA comprises a nucleotide sequence selected from the group consisting of SEQ ID NO: 3, SEQ ID NO 4 and SEQ ID NO: 9 and wherein hybridization of the antisense oligonucleotide with the BRHF1 mRNA reduces BRHF1 expression.

57. A method of reducing BRHF1 expression comprising administering to a cell a ribozyme that specifically binds to mRNA transcribed from the BRHF1 gene, the ribozyme binding reducing BRHF1 expression.

58. A method of reducing BRHF1 expression comprising administering to a cell an siRNA that is complementary to at least a portion of the coding sequence of BRHF1, under conditions that allow hybridization of the siRNA with the BRHF1 coding sequence, wherein the BRHF1 coding sequence comprises a nucleotide sequence selected from the group consisting of SEQ ID NO: 3, SEQ ID NO: 4 and SEQ ID NO: 9, and wherein the binding of the siRNA to the BRHF1 coding sequence reduces BRHF1 expression.

59. The method of claim 56, 57 or 58, wherein the cell is in a subject.

60. A method of identifying a compound that reduces BRHF1 expression, comprising administering a test compound to a cell containing a BRHF1 gene and detecting the level of the BRHF1 gene product produced, a decrease in the gene product as compared to a control cell indicating the compound reduces BRHF1 expression.

61. A method of identifying a compound that reduces BRHF1 expression, comprising administering a test compound to a cell containing a nucleic acid encoding the polypeptide of claim 21 and detecting the level of the BRHF1 gene product produced, a decrease in the gene product indicating a compound that reduces BRHF1 expression.

62. A method of identifying a compound that reduces BRHF1 expression in the presence of estrogen, comprising administering a test compound and estrogen to a cell containing a BRHF1 gene and detecting the level of the BRHF1 gene product produced, a decrease in the gene product as compared to a control cell indicating a compound that reduces BRHF1 expression in the presence of estrogen.

63. A method of identifying a compound that reduces BRHF1 expression in the presence of estrogen, comprising administering a test compound and estrogen to a cell containing a nucleic acid encoding the polypeptide of claim 21 and detecting the level of the BRHF1 gene product produced, a decrease in the gene product indicating a compound that reduces BRHF1 expression in the presence of estrogen.
